(19) United States
(12) Patent Application Publication
(10) Pub. No.: US 2006/0040868 A1
(43) Pub. Date: Feb. 23, 2006

(54) Methods for treating premature infants

(76) Inventor: Richard Lloyd Bowen, Raleigh, NC (US)

Correspondence Address: COVINGTON & BURLING;ATTN: PATENT DOCKETING, 1201 PENNSYLVANIA AVENUE, N.W., WASHINGTON, DC, 20004-2401, US

(21) Appl. No.: 11/200,112

(22) Filed: Aug. 10, 2005

Related U.S. Application Data
(60) Provisional application No. 60/599,852, filed on Aug. 10, 2004.

(51) Int. Cl. A61K 38/09

(52) U.S. Cl. 514/15

(57) ABSTRACT

Methods of treating premature infants include administering, to an infant, an agent that increases the blood or tissue levels, production, function, or activity of hCG, LH, FSH, GnRH, or activin or that decreases the blood or tissue levels, production, function, or activity of follistatin and inhibin.

This application claims the benefit, pursuant to 35 U.S.C. § 119, of U.S. Provisional Patent Application No. 60/599,852, filed Aug. 10, 2004, the entirety of which is incorporated herein by reference.

### FIELD OF THE INVENTION

The present invention relates to treating premature infants, and to improving the growth, differentiation, and development of premature infants and fetuses at risk for premature birth.

### BACKGROUND

Premature birth is a major public health concern, with approximately 476,000 premature births occurring in 2001 in the United States. The March of Dimes has estimated that the cost for medical care of premature babies is $13.6 billion per year in the United States.

Risk factors for premature delivery include prior occurrence of preterm birth, a pregnancy with multiple fetuses, age of the mother (less than 18 years of age or greater than 35 years of age), diabetes, hypertension, stress, and substance abuse (alcohol or drugs). Common problems related to immature organ systems in premature infants include, but are not limited to, respiratory distress syndrome, central nervous system immaturity that results in sucking and swallowing difficulty, susceptibility to bleeding in the brain, retinopathies, episodes of apnea, gastrointestinal immaturity that leads to feeding intolerance, cryptorchidism in male infants, and kidney immaturity. Depending on the severity of health problems in the infant, specialized medical care may be required for weeks, months, or even years due to long-lasting complications.

Many different therapies are in use currently to treat morbidities associated with premature infants. For example, infants at risk for or diagnosed with respiratory distress syndrome are candidates for surfactant administration, and preterm infants are commonly treated with surfactant to reduce alveolar surface tension in their lungs. Diuretics are used to improve pulmonary function since many preterm infants in respiratory distress display pulmonary edema. Further, massage therapy is being employed to increase weight gain in preterm infants. Extremely premature infants are given erythropoietin and iron supplements to prevent the need for erythrocyte transfusions. Trials are underway using Vitamin A administration to improve immune function, and immunoglobulin therapy is used to prevent nosocomial infections and to boost humoral immunity of the preterm infant.

Current hormonal therapies for preterm infants include antenatal administration of corticosteroids (dexamethasone, betamethasone) or postnatal administration of estradiol and progesterone, while corticosteroids are delivered to the mother to induce fetal lung maturation in anticipation of premature delivery. While repeated courses of antenatal steroids and high-dose postnatal dexamethasone appear to be deleterious to lung and brain development (Yeung M Y, Smyth J P. Hormonal factors in the morbidities associated with extreme prematurity and the potential benefits of hormonal supplement. Biology of the Neonate 81:1-15, 2002), single-dose antenatal corticosteroids are an effective treatment for respiratory distress syndrome (Celik C et al. Corticosteroid treatment for prevention of prematurity complications. Archives of Gynecology and Obstetrics 267:90-94, 2002). Administration of estradiol and progesterone to premature infants to replace that lost from the placental source was shown to slightly improve bone mineral accretion and to lessen the occurrence of chronic lung disease (Trotter A et al. Effects of postnatal estradiol and progesterone replacement in extremely preterm infants. Journal of Clinical Endocrinology and Metabolism 84:4531-4535, 1999).

### SUMMARY OF THE INVENTION

A problem with current treatments for premature infants is that most such treatments are aimed merely at the conditions and problems associated with prematurity. The treatments of the present invention, however, are aimed at the underlying problem of enhancing the developmental process in a way that mimics the growth and differentiation experienced by the fetus in utero under the influence of placental hormones.

The present invention proposes that hormones of the hypothalamic-pituitary-gonadal (HPG) axis are primarily responsible for the growth and development of the fetus and neonate, and that manipulating blood or tissue concentrations, production, function, or activity of these hormones during the antenatal period or in the preterm infant will improve the rate of growth and development of the fetus or infant, thereby decreasing the rate of morbidity and mortality.

According to this invention, administration, to the mother or fetus prior to birth or to the infant after birth, of agents that increase or regulate blood or tissue levels, production, function, or activity of gonadotropins (human chorionic gonadotropin (hCG), luteinizing hormone (LH), follicle stimulating hormone (FSH), or gonadotropin-releasing hormone (GnRH)) or that increase or regulate the function or activity of activin (either dimeric proteins or monomeric β-subunits), or that decrease or regulate blood or tissue levels, production, function, or activity of inhibin (either dimeric proteins or monomeric α-subunit) or follistatin, improves the growth, differentiation, and/or development of premature infants and fetuses at risk for premature birth.

In accordance with the present invention, an increase in the blood or tissue levels, production, function, or activity of hCG, LH, FSH, GnRH, or activin (either the dimeric proteins or the monomeric β-subunits) or a decrease in the blood or tissue levels, production, function, or activity of inhibin (either the dimeric proteins or monomeric α-subunit) or follistatin contributes to an increase in the rate of proliferation of cells or causes cells to differentiate (in effect, mature) in multiple organ systems in the premature infant, leading to improved thermoregulation, weight gain, improved lung function, improved digestive function, fewer complications from hyperbilirubinemia, decreased apneic episodes, less anemia, improved blood pressure, fewer bacterial, viral, and fungal infections, decreased intracerebral hemorrhages, and decreased severity of retinopathies.

In an embodiment of the invention, the blood or tissue levels, production, function, or activity of hCG, LH, FSH, or GnRH or the function or activity of activin (either the dimeric proteins or the monomeric β-subunits) are increased to levels that are as high as possible without causing significant adverse side effects. In another embodiment of the invention, the blood levels, production, function, or activity of inhibin (either the dimeric proteins or monomeric α-subunit) or follistatin are decreased to levels that are as low as possible without causing significant adverse side effects.

According to the invention, hCG, LH, FSH, GnRH, or activin and any analogues thereof are used to increase the blood or tissue levels, production, function or activity of these hormones. Agents that increase the blood or tissue levels, production, function or activity of hCG, LH, FSH, GnRH, or activin (either the dimeric proteins or the monomeric β-subunits) include but are not limited to recombinant or natural forms of these hormones, agents that stimulate production of these hormones, gene therapeutics that increase production of these hormones, gene therapeutics that decrease tissue or blood levels, or function, production, or activity of inhibitors of these hormones. An increase in the blood or tissue levels, production, function, or activity of hCG, LH, FSH, GNRH, or activin (either the dimeric proteins or the monomeric β-subunits) can also be achieved through active (vaccine) or passive immunization against inhibitors of these hormones, ribonucleic acid interference to prevent expression of proteins that inhibit these hormones, and dominant negative expression of genes that code for inhibitors of these hormones.

Agents that decrease the blood or tissue levels, production, function, or activity of follistatin and inhibin include but are not limited to vaccines that stimulate the production of antibodies that block the activity of follistatin or its binding site, vaccines that block the activity of inhibin (either the dimeric proteins or monomeric α-subunit) or its binding interaction with β-glycan, antibodies (passive immunization) that block the activity of follistatin (or its binding site) or inhibin (either the dimeric proteins or monomeric α-subunit), gene therapeutics including dominant negative expression of the genes which code for follistatin, inhibin (either the dimeric proteins or monomeric α-subunit), and β-glycan, ribonucleic acid interference directed at follistatin, inhibin (either the dimeric proteins or monomeric α-subunit), and β-glycan, and analogues of follistatin or small molecules or salts thereof that block the binding site of follistatin without inhibiting the function of activins.

Administration to the mother or fetus prior to birth or to the infant after birth of other agents, including agents not yet known, that increase or regulate blood levels, production, function, or activity of hCG, LH, FSH, or GnRH or the function or activity of activin (either the dimeric proteins or the monomeric β-subunits) or that decrease or regulate blood or tissue levels, production, function, or activity of inhibin (either the dimeric proteins or monomeric α-subunit) or follistatin is also encompassed within the present invention.

### DETAILED DESCRIPTION OF THE INVENTION

HYPOTHALAMIC-PITUITARY-GONADAL AXIS

The principal hormones responsible for regulating reproductive function include the centrally and peripherally produced hormones of the HPG axis. In humans and many other mammals, the centrally produced hormones include: gonadotropin releasing hormone (GnRH) from the hypothalamus and the placenta, human chorionic gonadotropin (hCG) from the placenta, and the gonadotropins luteinizing hormone (LH) and follicle stimulating hormone (FSH) from the pituitary. Peripherally produced hormones include estrogen, progesterone, testosterone, and inhibins that are primarily of gonadal origin, and activins and follistatin, which are produced in all tissues, including the gonads (Carr B R. In Wilson J D, Foster D W, Kronenberg H M, Larsen P R (eds): William's Textbook of Endocrinology, ed. 9. Philadelphia, Saunders, 1998, pp. 751-817).

The levels of each of these hormones are regulated by a complex feedback loop—GnRH secretion from the hypothalamus stimulates the anterior pituitary to secrete the gonadotropins, LH and FSH, which then bind to receptors in the gonads and stimulate oogenesis/spermatogenesis as well as sex steroid and inhibin production (Reichlin S. In Wilson J D, Foster D W, Kronenberg H M, Larsen P R (eds): William's Textbook of Endocrinology, ed. 9. Philadelphia, Saunders, 1998, pp. 165-248). The sex steroids then feed back to the hypothalamus and pituitary, resulting in a decrease in gonadotropin secretion (Thomer et al. In Wilson J D, Foster D W, Kronenberg H M, Larsen P R (eds): William's Textbook of Endocrinology, ed. 9. Philadelphia, Saunders, 1998, pp. 249-340).

Activins, which are produced in many tissues, also stimulate gonadotropin secretion (Ling et al. Pituitary FSH is released by a heterodimer of the beta-subunits from the two forms of inhibin. Nature 321:779-782, 1986; Vale et al. Purification and characterization of an FSH releasing protein from porcine ovarian follicular fluid. Nature 321:776-779, 1986). The stimulation of gonadotropin production by activins is inhibited by inhibins and follistatin. Inhibin binds to and inactivates activin receptors in a competitive manner. This inhibitory action is significantly enhanced in tissues whose cell membranes express β-glycan. Follistatin, on the other hand, directly and irreversibly binds to activins and prevents them from binding to their receptors (DeKretser D M et al. Inhibins, activins and follistatin in reproduction. Human Reproduction Update 8:529-541, 2002; Gray P C et al. Antagonism of activin by inhibin and inhibin receptors: a functional role for β-glycan. Molecular and Cellular Endocrinology 188:254-260, 2002).

Follistatin is expressed in many different tissues, and serum concentrations are known to change during pregnancy (Shang T et al. Concentrations of follistatin in maternal serum at term and its expression in the placenta. Zhonghua Fu Chan Ke Za Zhi 38:390-393, 2003) and puberty (Foster C M et al. Changes in serum inhibin, activin and follistatin concentrations during puberty in girls. Human Reproduction 15:1052-1057, 2000) as well as with certain medical conditions such as polycystic ovary syndrome (Eldar-Geva T et al. Relationship between serum inhibin A and B and ovarian follicle development after a daily fixed dose administration of recombinant follicle-stimulating hormone. Journal of Clinical Endocrinology and Metabolism 85:607-613, 2000; Thorner et al., In Wilson J D, Foster D W, Kronenberg H M, Larsen P R (eds): William's Textbook of Endocrinology, ed. 9. Philadelphia, Saunders, 1998, pp. 249-340). Follistatin also likely functions to regulate some of the non-reproductive actions of activins in an autocrine/paracrine fashion.

RELATIONSHIP BETWEEN HPG HORMONES AND GROWTH AND DEVELOPMENT

Starting with the fetal period, which is the time of greatest mitogenesis and tissue differentiation, most of the HPG hormones are significantly upregulated (Anderson A M et al. Longitudinal reproductive hormone profiles in infants: peak of inhibin B levels in infant boys exceeds levels in adult men. Journal of Clinical Endocrinology and Metabolism 83:675-681, 1998; Boyar R et al., Synchronization of augmented luteinizing hormone secretion with sleep during puberty. New England Journal of Medicine 287:582-586, 1972; Casey and MacDonald. In Wilson J D, Foster D W, Kronenberg H M, Larsen P R (eds): William's Textbook of Endocrinology, ed. 9. Philadelphia, Saunders, 1998, pp. 1259-1271; Fisher D A. In Wilson J D, Foster D W, Kronenberg H M, Larsen P R (eds): William's Textbook of Endocrinology, ed. 9. Philadelphia, Saunders, 1998, pp. 1273-1301). hCG and LH have similar sequence homology and share a common receptor to which they bind with similar affinity (Fiddes and Talmadge. Structure, expression, and evolution of the genes for the human glycoprotein hormones. Recent Progress in Hormone Research 40:43-78, 1984). During fetal life, maternal LH/hCG concentrations are up to 5,000 times higher than at any other time of life, and these hormones are known to cross into fetal circulation, albeit at lower concentrations. (Casey and MacDonald. In Wilson J D, Foster D W, Kronenberg H M, Larsen P R (eds): William's Textbook of Endocrinology, ed. 9. Philadelphia, Saunders, 1998, pp. 1259-1271).

Fetal serum concentrations of progesterone, inhibins, activins, hCG, and FSH decrease at birth with the loss of the placenta (Fisher D A In Wilson J D, Foster D W, Kronenberg H M, Larsen P R (eds): William's Textbook of Endocrinology, ed. 9. Philadelphia, Saunders, 1998, pp. 1273-1301), but these hormones, except for progesterone, begin to rise within approximately two weeks (Boyar et al. Synchronization of augmented luteinizing hormone secretion with sleep during puberty. New England Journal of Medicine 287:582-586, 1972; Grumbach M M, Styne D M. In Wilson J D, Foster D W, Kronenberg H M, Larsen P R (eds): William's Textbook of Endocrinology, ed. 9. Philadelphia, Saunders, 1998, pp. 1115-1286, 1998) corresponding to the growth of the human newborn. Infants lose weight initially and do not begin to grow again until the second week of life. (Itabashi K et al. Postnatal growth curves of very low birth weight Japanese infants. Acta Paediatrica Japan 34:648-655, 1992; Smith SL et al. Patterns of postnatal weight changes in infants with very low and extremely low birth weights. Heart and Lung 23:439-445, 1994). LH/hCG, FSH, inhibins, and activins then continue to rise, peaking at approximately 3 months of age, and thereafter begin to decline, reaching childhood levels by 9 months of age (Thorner et al. In Wilson J D, Foster D W, Kronenberg H M, Larsen P R (eds): William's Textbook of Endocrinology, ed. 9. Philadelphia, Saunders, 1998, pp. 249-340). This pattern of reproductive hormone secretion also mirrors the rapid rate of growth (mitogenesis) and development (differentiation) during the first year of life. Serum concentrations of these hormones, as well as growth and development, remain comparatively diminished throughout the rest of childhood until the onset of puberty (Thorner et al. In Wilson J D, Foster D W, Kronenberg H M, Larsen P R (eds): William's Textbook of Endocrinology, ed. 9. Philadelphia, Saunders, 1998, pp. 249-340).

With the onset of puberty, there is an increase in the secretion of all HPG hormones (Grumbach and Styne. In Wilson J D, Foster D W, Kronenberg H M, Larsen P R (eds): William's Textbook of Endocrinology, ed. 9. Philadelphia, Saunders, 1998, pp. 1115-1286). Some of these hormones likely contribute significantly to the rapid increase in the rate of growth (mitogenesis), while others may be responsible for the developmental (differentiation) changes experienced during puberty. The completion of puberty marks the end of growth and development.

Evidence supporting a role for FSH, LH, and hCG in driving cell proliferation includes the following: 1) FSH is associated with granulosa cell proliferation (El-Heffiawy T, Zeleznik A J. Synergism between FSH and activin in the regulation of proliferating cell nuclear antigen (PCNA) and cyclin D2 expression in rat granulosa cells. Endocrinology 142:4357-4362, 2001); 2) hCG directly promotes the proliferation of myometrial and leiomyomal cells (Horiuchi A et al. HCG promotes proliferation of uterine leiomyomal cells more strongly than that of myometrial smooth muscle cells in vitro. Molecular Human Reproduction 6:523-528, 2000); 3) the basal proliferation of ovarian surface epithelium can be significantly increased by administration of pure recombinant gonadotropins FSH or LH (Davies B R et al. Administration of gonadotropins stimulates proliferation of normal mouse ovarian surface epithelium. Gynecology and Endocrinology 13:75-81, 1999); 4) one study also has shown that LH stimulates the growth of chondrocytes (cartilage cells) in rabbit epiphyseal growth plates (Webber R J, Sokoloff L. In vitro culture of rabbit growth plate chondrocytes: 1. Age-dependence of response to fibroblast growth factor and “chondrocyte growth factor.” Growth 45:252-268, 1981); and 5) unpublished work demonstrated that growth of cultured M-17 neuroblastoma cells was induced by LH (Bowen et al., unpublished observations). The mechanism by which these hormones exert their mitogenicity is likely via signaling through the insulin/IGF pathway that converges on FKHR (human homolog of daf-16), phosphorylation of which stimulates mitosis (Richards, J S, Sharma, S C, Falender, A E, Lo, Y H. Expression of FKHR, FKHRL1, and AFX genes in the rodent ovary: evidence for regulation by IGF-I, estrogen, and the gonadotropins. Molecular Endocrinology 16, 580-599, 2002). This is based on the following: recent evidence that FSH and LH regulate FKHR transcription (Hsu S Y, Liang, S G, Hsueh A J. Characterization of two LGR genes homologous to gonadotropin and thyrotropin receptors with extracellular leucine-rich repeats and a G-protein-coupled, seven-transmembrane region. Molecular Endocrinology 12:1830-1845, 1998, Richards J S, Sharma S C, Falender A E, Lo Y H. Expression of FKHR, FKHRL1, and AFX genes in the rodent ovary: evidence for regulation by IGF-I, estrogen, and the gonadotropins. Molecular Endocrinology 16:580-599, 2002); LH has been shown to increase signaling via the PI3K/AKT pathway (Oust as IGF-1 does) (Carvalho C R, Carvalheira J B, Lima M H, Zimmerman S F, Caperuto L C, Amanso A, Gasparetti A L, Meneghetti V, Zimmerman L F, Velloso L A, Saad M J. Novel signal transduction pathway for luteinizing hormone and its interaction with insulin: activation of Janus kinase/signal transducer and activator of transcription and phosphoinositol 3-kinase/Akt pathways. Endocrinology 144, 638-647, 2003) that is known to phosphorylate FKHR; and in naive rodent granulosa cells, both FSH and IGF-1 stimulate rapid phosphorylation of FKHR at multiple sites, causing its redistribution from the nucleus to the cytoplasm in a PI3K-dependent manner, thereby initiating mitogenesis (Biggs W H 3rd, Meisenhelder J, Hunter T, Cavenee W K, Arden K C. Protein kinase B/Akt-mediated phosphorylation promotes nuclear exclusion of the winged helix transcription factor FKHR1. Proc Natl Acad Sci USA 96, 7421-7426, 1999). Additionally, in differentiated granulosa cells, FSH enhances phosphorylation of FKHR, PKB, and Sgk (Richards J S, Sharma S C, Falender A E, Lo Y H. Expression of FKHR, FKHRL1, and AFX genes in the rodent ovary: evidence for regulation by IGF-I, estrogen, and the gonadotropins. Mol Endocrinol 16, 580-599, 2002).

While this invention proposes that hCG, GnRH, LH, and FSH are likely to be mitogenic factors driving growth (cell proliferation), it also proposes that activins likely represent differentiation factors that allow for cells to differentiate and perform the unique functions required for a newborn organism to survive. This is based on the fact that while activins have been shown to both stimulate and inhibit cell proliferation in reproductive and non-reproductive tissues, in most tissues they function to promote differentiation (Asashima M, Ariizumi T, Malacinski G M. In vitro control of organogenesis and body patterning by activin during early amphibian development. Comp Biochem Physiol B Biochem Mol Biol 126, 169-178, 2002). Activins have been shown to be important in tissue differentiation during fetal development in that they are required for endometrial receptivity, decidualization, and implantation (Jones R L, Salamonsen L A, Findlay J K. Activin A promotes human endometrial stromal cell decidualization in vitro. J Clin Endocrinol Metab 87, 4001-4004, 2002). Moreover, activins regulate follicular development (Roberts V J, Barth S, el-Roeiy A, Yen S S. Expression of inhibin/activin subunits and follistatin messenger ribonucleic acids and proteins in ovarian follicles and the corpus luteum during the human menstrual cycle. J Clin Endocrinol Metab 77, 1402-1410, 1993). Given that all cell types undergo differentiation, it would be expected that the receptors for the differentiation factor would be expressed in most tissues, and such is the case with activin receptors (Baer H, Friess H, Abou-Shady M, Berberat P, Zimmermann A, Gold L, Korc M, Buchler M. Transforming growth factor betas and their receptors in human liver cirrhosis. Eur J Gastroenterol Hepatol 10, 1031-1039, 1998; Baldwin R L, Friess H, Yokoyama M, Lopez M E, Kobrin M S, Buchler M W, Korc M. Attenuated ALK5 receptor expression in human pancreatic cancer: correlation with resistance to growth inhibition. Int J Cancer 67, 283-288, 1996; Dewulf N, Verschueren K, Lonnoy O, Moren A, Grimsby S, VandeSpiegle K, Miyazono K, Huylebroeck D, TenDijke P. Distinct spatial and temporal expression patterns of two type I receptors for bone morphogenetic proteins during mouse embryogenesis. Endocrinology 136, 2652-2663, 1995; Kitten A M, Kreisberg J I, Olson M S. Expression of osteogenic protein-1 mRNA in cultured kidney cells. J Cell Physiol 181, 410-415, 1999; Li G, Borger M A, Williams W G, Weisel R D, Mickle D A, Wigle E D, Li R K. Regional overexpression of insulin-like growth factor-I and transforming growth factor-beta1 in the myocardium of patients with hypertrophic obstructive cardiomyopathy. J Thorac Cardiovasc Surg 123, 89-95, 2002; Schluns K S, Grutkoski P S, Cook J E, Engelmann G L, Le P T. Human thymic epithelial cells produce TGF-beta 3 and express TGF-beta receptors. Int Immunol 7, 1681-1690, 1995).

LUTEINIZING HORMONE

LH is a member of the pituitary gonadotropin family of glycoprotein hormones that includes FSH, thyroid stimulating hormone (TSH), and the placentally derived hCG. These hormones are heterodimers of a common cesubunit with a unique β-subunit. The complementary DNAs and genes for the α-subunit have been characterized in human, mouse, and rat. The α-subunit is composed of 4 exons and 3 introns, and there is considerable variation in the length of intron 1 between species. A small mRNA of ˜800 nucleotides is produced in all species, and the resulting hormone consists of 92 amino acids. In humans, the amino acid similarity between LH and hCG β-subunits is 82%. The LH β-subunit is synthesized in the pituitary gonadotroph cells, while hCG β-subunit is synthesized in the syncytiotrophoblast of the placenta.

LH and hCG share a common receptor, the luteinizing hormone receptor (LHR), which is a single polypeptide chain and shares structural similarities with the rhodopsin/β2-adrenergic receptor subfamily of G protein-coupled receptors (Gether U. Uncovering molecular mechanisms involved in activation of G protein-coupled receptors. Endocrine Reviews 21:90-113 (2000)). The mature human LHR consists of 675 amino acids and has three distinct domains: an N-terminal extracellular domain, a serpentine domain with seven transmembrane segments connected by three extracellular loops and three intracellular loops, and an intracellular C-terminal tail (Ascoli M, Fanelli F, and Segaloff, D L. The lutropin/choriogonadotropin receptor, a 2002 perspective. Endocrine Reviews 23:141-174 (2002)). LHR is expressed primarily in testicular Leydig cells and ovarian theca, interstitial, differentiated granulosa and luteal cells but has also been reported in a variety of other tissues including uterus (Reshef E, Lei Z M, Rao C V, Pridham D D, Chegini N, Luborsky J L. The presence of gonadotropin receptors in nonpregnant human uterus, human placenta, fetal membranes, and deciduas. Journal of Clinical Endocrinology and Metabolism 70:421-430 (1990)), human sperm, human seminal vesicles, rat and human prostate, human prostate cancer, skin, breast cell lines, lactating rat mammary gland, human adrenals, neural retina, neuroendocrine cells, and rat brain (reviewed in Ascoli M, Fanelli F, Segaloff, D L. The lutropin/choriogonadotropin receptor, a 2002 perspective. Endocrine Reviews 23:141-174, 2002). Hormone binding to the LHR extracellular domain leads to activation of two G protein-dependent signaling pathways, adenylyl cyclase/cAMP/protein kinase A and phospholipase C (PLC), leading to subsequent signaling through the PI3 kinase/Akt pathway that induces mitogenesis.

FOLLICLE STIMULATING HORMONE

A single gene with three exons and two introns encodes the FSH β-subunit. FSH is produced by pituitary gonadotroph cells and acts by binding to specific receptors, localized exclusively in the gonads. The FSH receptor shares a similar structure with LHR and belongs to the family of G protein-coupled receptors. FSH binds specifically to receptors on Sertoli cells in the testis and on granulosa cells in the ovary (reviewed in Simoni M, Gromoll J, Nieschlag E. The follicle-stimulating hormone receptor: Biochemistry, molecular biology, physiology, and pathophysiology. Endocrine Reviews 18:739-773, 1997).

GONADOTROPIN RELEASING HORMONE

GnRH is a hypothalamic neuropeptide consisting of ten amino acids. Two genes encode GnRH: GnRH-I is found in hypothalamic neurons and serves as a releasing factor to regulate pituitary gonadotroph function, and GnRH-II encodes a decapeptide similar to GnRH-I, with the exception of three amino acid substitutions, that acts as a neurotransmitter in the midbrain. GnRH binds to a membrane receptor on pituitary gonadotrophs and stimulates production and release of LH and FSH. The GnRH receptor is a G protein-coupled receptor but lacks an intracellular C-terminal cytoplasmic domain. Upon activation, the GNRH receptor couples to phospholipase C, which leads to increases in calcium influx into gonadotroph cells and calcium release from internal stores through the action of a diacylglycerol-protein kinase C pathway. Mitogen activated protein (MAP) kinase signaling is also activated by GnRH (reviewed in Cone R D, Low M J, Elmquiest J K, Camerson J L. Neuroendocrinology. In Wilson J D, Foster D W, Kronenberg H M, Larsen P R (eds): William's Textbook of Endocrinology, ed. 9. Philadelphia, Saunders, 1998, pp. 81-176).

LH/HCG, FSH AND GNRH AS GROWTH PROMOTING/CELL DIFFERENTIATION FACTORS

The present invention proposes that maternally produced hCG and GnRH play a direct functional role in fetal development. In preterm infants, the loss of the placentally-produced LH/hCG or GnRH at the time of delivery contributes to the delayed growth and development of the newborn. Therefore, administering these hormones to the mother or fetus prior to birth or to the infant after birth will increase the rate of growth and development and decrease the morbidity associated with preterm birth. The invention is supported by the fact that even in full term infants, their rate of growth and development correlates to serum concentrations of GnRH, LH, and FSH. At birth, with the loss of the placenta, levels of these hormones are low, and the infant begins to lose weight. This weight loss normally continues for ten to fourteen days, at which time the infant starts gaining weight. This is precisely the time that serum gonadotropin concentrations begin to rise. In preterm infants, the greater the degree of prematurity, the greater the duration of postnatal weight loss (Ehrenkranz R A et al. Longitudinal growth of hospitalized very low birth weight infants. Pediatrics 104:280-289, 1999; Pauls J. et al. Postnatal body weight curves for infants below 1000 g birth weight receiving enteral and parenteral nutrition. European Journal of Pediatrics 157:416-421, 1998). It is interesting to note that the degree of prematurity also corresponds to the length of time it takes for gonadotropins to begin to rise.

The mitogenic/differentiation properties of these hormones may also explain the gender difference in the mortality and morbidity of preterm infants. The serum concentrations of these hormones are very different between preterm males and females. Serum FSH levels in cord serum from preterm females (5.4±1.8 IU/L) was shown to be significantly higher than in males (1.5±0.08 IU/L), and decreased in preterm females towards full term. During the first 10 postnatal weeks, when preterm infant growth is slow compared to full term infant growth, serum FSH is 10-20 times higher and serum LH is 3-4 times higher in premature than in fullterm girls whereas these differences were not observed in boys (Tapanainen J et al. Hormonal changes during the perinatal period: FSH, prolactin and some steroid hormones in the cord blood and peripheral serum of preterm and fullterm female infants. Journal of Steroid Biochemistry 20:1153-56, 1984). FSH levels in female infants increased to peak levels between 11 and 30 days after delivery and then decreased, and this elevated level was prolonged in preterm infants compared to normal term infants (Shinkawa O et al. Changes of serum gonadotropin levels and sex differences in premature and mature infant during neonatal life. Journal of Clinical Endocrinology and Metabolism 56:1327-1331, 1983). In preterm infants (gestational age 26-32 weeks), inhibin and LH levels were higher in males compared to females. At term birth, FSH and LH levels were undetectable (Massa G et al. Serum levels of immunoreactive inhibin, FSH, and LH in human infants at preterm and term birth. Biology of the Neonate 61:150-155, 1992).

The invention proposes that at any given weight, the female infant is more developed than the male infant. This is true for verbal development, with female brains having denser concentrations of neurons in the cerebral cortex. Females enter puberty two years earlier than males, and when fully developed, have a significantly lower lean body mass (Grumbach, M, Styne, D M. Puberty: Ontogeny, neuroendocrinology, physiology, and disorders. In Wilson J D, Foster D W, Kronenberg H M, Larsen P R (eds): William's Textbook of Endocrinology, ed. 9. Philadelphia, Saunders, 1998). Therefore, even though males and females may be similar in size, the female is further along in the developmental process. Unfortunately for males, who grow faster, it appears that parturition is dependent on the ability of the placenta to support a particular fetal mass. Studies have demonstrated that fetal growth depends on the actual weight of the placenta (Heionen S et al. Weights of placentae from small-for-gestational age infants revisited. Placenta 22:399-404, 2001) and that placental volume in the second trimester predicted birth size more accurately than fetal measurements (Thame M et al. Second-trimester placental volume and infant size at birth. Obstetrics and Gynecology 98:279-283, 2001). This suggests that a rate-limiting factor in fetal development is the ability of the mother to generate a placenta of sufficient volume to enable full-term gestation. Hormonal signals from the fetus may regulate this process, and the invention proposes that hCG and GnRH are two important factors in continued fetal growth in utero.

ACTIVINS

While cell proliferation is responsible for fetal growth, it is cell differentiation that is responsible for fetal development. The invention proposes that GnRH and hCG drive cell proliferation which then leads to cell differentiation. This differentiation is due to increasing concentrations of particular activins which are members of the TGF-β family of proteins and are well known to function in this role. Therefore, by administering particular activins to the mother or fetus prior to birth or to the infant after birth, it will be possible to increase the rate of development of specific vital organ tissues, such as the lungs, thereby minimizing the associated morbidity.

The manner by which activins (Nishimura R et al. Smad5 and DPC4 are key molecules in mediating BMP-2-induced osteoblastic differentiation of the pluripotent mesenchymal precursor cell line C2C12. Journal of Biological Chemistry 273:1872-1879, 1998) affect cellular function is extremely complex in that there are at least five different activin receptors, and these receptors share the same post-receptor signaling mechanism with at least seven other bone morphogenetic protein receptors (reviewed in Kawabata, M et al. Signal transduction by bone morphogenetic proteins. Cytokine and Growth Factor Reviews 9:49-61, 1998; Miyazono, K. Positive and negative regulation of TGF-beta signaling. Journal of Cell Science 113:1101-1109, 2000) and by phosphorylating up to eight different Smad proteins (Hoodless P A et al. MADRI1, a MAD-related protein that functions in BMP2 signaling pathways. Cell 85:489-500, 1996; Kawai S et al. Mouse smad8 phosphorylation downstream of BMP receptors ALK-2, ALK-3, and ALK-6 induces its association with Smad4 and transcriptional activity. Biochemical and Biophysical Research Communications 271:682-687, 2000; Nishimura R et al. Smad5 and DPC4 are key molecules in mediating BMP-2-induced osteoblastic differentiation of the pluripotent mesenchymal precursor cell line C2C12. Journal of Biological Chemistry 273:1872-1879, 1998). Smads then participate directly in the regulation of gene expression by binding to DNA, interacting with transcription factors, and recruiting corepressors or coactivators to specific promoters (van Grunsven L A et al. Complex Smad-dependent transcriptional responses in vertebrate development and human disease. Critical Reviews in Eukaryotic Gene Expression 12:101-118, 2002).

A further example of this complexity is exemplified by activin subunit interactions with one another. Activins and inhibins are dimeric proteins consisting of two non-covalently linked subunits which include one α subunit and/or five β subunits; A, B, C, D, and E (Fang J et al. Molecular cloning of the mouse activin beta E subunit gene. Biochemical and Biophysical Research Communications 228:669-674, 1996; Hotten G C et al. Recombinant human growth/differentiation factor 5 stimulates mesenchyme aggregation and chondrogenesis responsible for the skeletal development of limbs. Growth Factors 13:65-74, 1996; Oda S et al. Molecular cloning and functional analysis of a new activin beta subunit: a dorsal mesoderm-inducing activity in Xenopus. Biochemical and Biophysical Research Communications 210:581-588, 1995; Vale W et al. In Peptide Growth Factors and Their Receptors. Sporn M B, Roberts A B (Heidelberg, Germany, Springer-Verlag), pp. 211-248, 1991). The α-subunit is expressed primarily in reproductive tissues and is directly correlated to oogenesis and spermatogenesis, while β-subunits are expressed in reproductive and numerous other tissues (Hubner G et al. Activin: a novel player in tissue repair processes. Histology and Histopathology 14:295-304, 1999). Inhibin A is composed of an αsubunit and a βA subunit. Inhibin B consists of an α subunit and a βB subunit (Bernard D J et al. Mechanisms of inhibin signal transduction. Recent Progress in Hormone Research 56:417-450, 2001). Activin A is composed of two βA subunits, activin AB is composed of one βA and one βB subunits, and activin B is composed of two βB subunits (Halvorson L M, DeCherney A H. Inhibin, activin, and follistatin in reproductive medicine. Fertility and Sterility 65:459-469, 1996). Since β-subunits C, D and E have only recently been identified, very little is known about their interactions with the other subunits (Hotten G C et al. Recombinant human growth/differentiation factor 5 stimulates mesenchyme aggregation and chondrogenesis responsible for the skeletal development of limbs. Growth Factors 13:65-74, 1996; Mellor S L et al., Localization of activin beta(A)-, beta(B)-, and beta(C)-subunits in human prostate and evidence for formation of new activin heterodimers of beta(C)-subunit. Journal of Clinical Endocrinology and Metabolism 85:4851-4858, 2000; O'Bryan M K et al. Cloning and regulation of the rat activin betaE subunit. Journal of Molecular Endocrinology 24:409-418, 2000). Activins bind to specific receptors in the serine/threonine bone morphogenetic protein receptor family which, as mentioned previously, are expressed in all tissues thus far examined (Ethier J F, Findlay J K Roles of activin and its signal transduction mechanisms in reproductive tissues. Reproduction 121:667-675, 2001). It remains to be determined if there are unique inhibin receptors; inhibin has, however, been shown to bind to ActRII's (Zimmerman C M, Mathews L S. Activin receptors and their mechanism of action. In: Inhibin, Activin and Follistatin in Hyman Reproductive Physiology. Muttukrishna S., Ledger W (eds), London, England, Imperial College Press, 2001, pp. 239-277). It appears that inhibins function primarily to regulate the activity of activins by binding the activin receptor, thereby preventing its activation by activins (Bernard D J et al. Mechanisms of inhibin signal transduction. Recent Progress in Hormone Research 56:417-450, 2001). Even further complexity is evidenced by the fact that the receptor affinity of inhibins is greatly influenced by the presence or absence of the β-glycan content of the cell membrane.

The role of β:β dimers (activins) in regulating differentiation is well established by numerous studies in a wide range of species and tissues (Chertov O et al. Mesoderm-inducing factor from bovine amniotic fluid: purification and N-terminal amino acid sequence determination. Biomedical Sciences 1:499-506, 1990; Dirksen M L, Jamrich M. A novel, activin-inducible, blastopore lip-specific gene of Xenopus laevis contains a fork head DNA-binding domain. Genes and Development 6:599-608, 1992; Kokan-Moore N P et al. Secretion of inhibin beta A by endoderm cultured from early embryonic chicken. Developmental Biology 146:242-245, 1991; Strahle U et al. Axial, a zebrafish gene expressed along the developing body axis, shows altered expression in cyclops mutant embryos. Genes and Development 7:1436-1446, 1993).

In pregnant women, serum activin A levels were shown to increase in the final month of normal pregnancy, whereas activin B was undetectable. Total serum follistatin increased 10-45 fold in the final month of normal pregnancy in a subset of women and returned to basal serum concentrations in a separate group of women during the last two weeks of pregnancy. Activin A production exceeded the binding capacity of circulating follistatin, suggesting that activin A detected late in pregnancy is important for normal labor and development of the fetus (Woodruff T K et al. Activin A and follistatin are dynamically regulated during human pregnancy. Journal of Endocrinology 152:167-174, 1997). Another study demonstrated that the concentration of activin A and inhibin A increased with gestational age in maternal serum (Keelan J A et al. Serum activin A, inhibin A, and follistatin concentrations in preeclampsia or small for gestational age pregnancies. Obstetrics and Gynecology 99: 267-274, 2002). While inhibin B remains nearly undetectable during gestation, maternal inhibin A increases 5-6 fold from 28 to 36 weeks of gestation (Muttukrishna S et al. Measurement of serum concentrations of inhibin-A (α-β_{A} dimer) during human pregnancy. Clinical Endocrinology 42:391-397, 1995).

An embodiment of the present invention includes administering an agent to the mother or fetus prior to birth or to the infant after birth that increases or regulates the blood or tissue levels, production, function, or activity of hCG, LH, FSH, or GnRH or increases or regulates the function or activity of activin (either the dimeric proteins or the monomeric β-subunits) or decreases or regulates the blood levels, production, function, or activity of inhibin (either the dimeric proteins or monomeric α-subunit) or follistatin to blood or tissue levels, production, function, or activity similar to that occurring at the corresponding gestational age of a full term infant.

In another embodiment, the present invention encompasses administering an agent to the mother or fetus prior to birth or to the infant after birth that increases or regulates blood or tissue levels, production, function, or activity of hCG, LH, FSH, or GnRH or increases or regulates the function or activity of activin (either the dimeric proteins or the monomeric β-subunits) to blood or tissue levels, production, function, or activity that are approximately as high as possible without causing significant adverse side effects.

In a further embodiment, the present invention encompasses administering an agent to the mother or fetus prior to birth or to the infant after birth that decreases or regulates the levels, production, function, or activity of inhibin (either the dimeric proteins or monomeric α-subunit) or follistatin to blood or tissue levels, production, function, or activity that are approximately as low as possible without causing significant adverse side effects.

In other embodiments of the present invention, the blood or tissue levels, production, function, or activity of hCG, LH, FSH, or GnRH or the function or activity of activin (either the dimeric proteins or the monomeric β-subunits) are continuously increased or regulated, or the blood levels, production, function, or activity of inhibin (either the dimeric proteins or monomeric α-subunit) or follistatin are continuously decreased or regulated, by monitoring the blood levels, production, function, or activity and making adjustments to the agents being administered via a feedback control system.

Fetal growth occurs throughout gestation, but the rate is highest between 24-28 weeks. Preterm delivery and subsequent extrauterine stresses prevent the premature infant from achieving this accelerated growth velocity. Although all infants experience weight loss following delivery due to fluid shifts between intracellular and extracellular compartments, growth patterns for preterm infants with birth weights less than 1000 grams are characterized by a longer period to regain birth weight and slower growth velocity compared to normal birth weight full term infants (Ehrenkranz, R A et al. Longitudinal growth of hospitalized very low birth weight infants. Pediatrics 104:280-289, 1999; Pauls, J et al. Postnatal body weight curves for infants below 1000 g birth weight receiving enteral and parenteral nutrition. European Journal of Pediatrics 157:416-421, 1998).

What is claimed is:

1. A method of treating premature infants comprising the step of administering, to an infant, a pharmaceutically effective amount of an agent that increases blood or tissue levels, production, function, or activity of hCG, LH, FSH, GnRH, or activin.

2. The method of claim 1, wherein the agent is LH or hCG.

3. The method of claim 1, wherein in the administering step, the pharmaceutically effective amount of the agent is administered to the mother of the infant prior to birth of the infant.

4. The method of claim 1, wherein in the administering step, the pharmaceutically effective amount of the agent is administered directly to the infant before or after birth of the infant.

5. A method of treating premature infants comprising the step of administering, to an infant, a pharmaceutically effective amount of an agent that decreases blood or tissue levels, production, function, or activity of follistatin or inhibin.

6. The method of claim 5, wherein in the administering step, the pharmaceutically effective amount of the agent is administered to the mother of the infant prior to birth of the infant.

7. The method of claim 5, wherein in the administering step, the pharmaceutically effective amount of the agent is administered directly to the infant before or after birth of the infant.

8. A method of treating one or more diseases or conditions associated with infant prematurity comprising the step of administering, to an infant, a pharmaceutically effective amount of an agent that increases blood or tissue levels, production, function, or activity of hCG, LH, FSH, GnRH, or activin.

9. The method of claim 8, wherein the agent is LH or hCG.

10. The method of claim 8, wherein in the administering step, the pharmaceutically effective amount of the agent is administered to the mother of the infant prior to birth of the infant.

11. The method of claim 8, wherein in the administering step, the pharmaceutically effective amount of the agent is administered directly to the infant before or after birth of the infant.

12. The method of claim 8, wherein the one or more diseases or conditions associated with infant prematurity is at least one of respiratory distress syndrome, central nervous system immaturity that results in sucking and swallowing difficulty, susceptibility of bleeding in the brain, retinopathies, episodes of apnea, gastrointestinal immaturity that leads to feeding intolerance, cryptorchidism in male infants, and kidney immaturity.

13. A method of treating one or more diseases or conditions associated with infant prematurity comprising the step of administering, to an infant, a pharmaceutically effective amount of an agent that decreases blood or tissue levels, production, function, or activity of follistatin or inhibin.

14. The method of claim 13, wherein in the administering step, the pharmaceutically effective amount of the agent is administered to the mother of the infant prior to birth of the infant.

15. The method of claim 13, wherein in the administering step, the pharmaceutically effective amount of the agent is administered directly to the infant before or after birth of the infant.

16. The method of claim 13, wherein the one or more diseases or conditions associated with infant prematurity is at least one of respiratory distress syndrome, central nervous system immaturity that results in sucking and swallowing difficulty, susceptibility of bleeding in the brain, retinopathies, episodes of apnea, gastrointestinal immaturity that leads to feeding intolerance, cryptorchidism in male infants, and kidney immaturity.
